(19) United States
(12) Patent Application Publication
(10) Pub. No.: US 2025/0041244 A1
(43) Pub. Date: Feb. 6, 2025

(54) PHARMACEUTICAL COMPOSITION COMPRISING SALBUTAMOL

(71) Applicant: APTAR FRANCE SAS, Le Neubourg (FR)

(72) Inventors: Isabelle PEYRON, Castres (FR); Irene ROSSI, San Benedetto del Tronto (IT); Ségolène SARRAILH, Heudebouville (FR)

(21) Appl. No.: 18/719,982

(22) PCT Filed: Dec. 19, 2022
(86) PCT No.: PCT/FR2022/052421

§ 371 Date: Jun. 14, 2024

(30) Foreign Application Priority Data
Dec. 20, 2021 (FR) FR2113999

(51) Int. Cl. A61K 31/137; A61K 9/00; A61K 47/06; A61K 47/10; A61K 47/12

(52) U.S. Cl. CPC A61K 31/137; A61K 9/008; A61K 47/06; A61K 47/10; A61K 47/12

(57) ABSTRACT

A pharmaceutical composition consisting of an active ingredient based on a pharmaceutically acceptable salbutamol salt, 1,1-difluoroethane (R-152a) and ethanol. The use thereof in the treatment of respiratory disorders, to a canister comprising it as well as to a metered-dose inhaler provided with such a canister. Finally, uses of this pharmaceutical composition and of this canister in a metered-dose inhaler.

### TECHNICAL FIELD

The present invention relates to a pharmaceutical composition that is capable of being used in the treatment of respiratory disorders and which comprises an active ingredient based on a salbutamol salt, in particular salbutamol sulphate, and a propellant gas formed by 1,1-difluoroethane.

The invention also relates to a canister comprising this pharmaceutical composition as well as to a metered-dose inhaler provided with such a canister.

The invention finally relates to the uses of this pharmaceutical composition and of this canister in a metered-dose inhaler.

### STATE OF THE PRIOR ART

Salbutamol as well as its derivatives are active ingredients known as bronchodilators in the treatment of respiratory disorders such as asthma and chronic obstructive pulmonary disease (COPD).

Pharmaceutical compositions comprising salbutamol or one of its derivatives are conventionally delivered to patients by means of a metered-dose inhaler (MDI).

A metered-dose inhaler is an administration device equipped with a canister containing the pharmaceutical composition, a metering valve allowing to distribute a controlled quantity of pharmaceutical composition containing the active ingredient and an applicator allowing to exert a pressure on the metering valve and fitted with a mouthpiece.

The pharmaceutical composition comprises a propellant gas in which the active ingredient is dissolved, suspended or dispersed, and optionally one or more other compounds that can in particular be chosen from surfactants, polar excipients and preservatives.

The choice of the propellant gas implemented in the metered-dose inhalers for pharmaceutical purposes has changed over the years.

Given their detrimental effects on the ozone layer, chlorofluorocarbons (CFC), used for a long time, were abandoned in favour of hydrofluorocarbons (HFC), more recently designated by the term hydrofluoroalkanes (HFA), such as 1,1,1,2-tetrafluoroethane (HFC-134a, HFA-134a or R-134a) and 1,1,1,2,3,3,3-heptafluoropropane (HFC-227ea, HFA-227ea or R-227ea) which are compounds that do not have a harmful effect on the ozone layer or human toxicity.

However, since these hydrofluoroalkanes R-134a and R-227ea are characterised by a high global warming potential (GWP) with a non-negligible impact on the greenhouse effect, pharmaceutical compositions comprising salbutamol, or one of its derivatives, and an alternative propellant gas have been proposed.

Thus, documents WO 2013/054137 A1, WO 2014/170689 A1 and WO 2013/054135 A1, respectively referred to as [1] to [3] in the rest of the present description, describe the implementation of a particular hydrofluorocarbon (HFC) or hydrofluoroalkane (HFA), 1,1-difluoroethane (HFC-152a, HFA-152a or R-152a), in pharmaceutical compositions comprising salbutamol sulphate.

### Document US 2007/041911 A1, Referred to as [4], Incidentally Cites

HFC-152a among a certain number of hydrofluorocarbons capable of being used as a propellant gas in pharmaceutical compositions comprising an acid addition salt of salbutamol, a co-solvent as well as an organic or inorganic acid. However, document [4] does not describe any example of a pharmaceutical composition specifically implementing R-152a.

Recent document US 2021/244688 A1, referred to as [5], describes pharmaceutical compositions comprising salbutamol, alone or in association with at least one long-acting muscarinic antagonist and/or at least one corticosteroid, as well as HFA-152a as propellant gas. However, in document [5], the salbutamol implemented is a so-called salbutamol “base”, which is defined as excluding all the pharmaceutically acceptable derivatives of salbutamol, in particular the salts of salbutamol.

More particularly, the pharmaceutical compositions described in documents [1] and [2] comprise salbutamol sulphate, R-152a as well as one or more surfactants, the role of which is to aid in the dispersion of the particles of active ingredient in the propellant gas.

In document [1], this surfactant is oleic acid whereas in document [2], this surfactant comprises at least one compound other than oleic acid.

Although these are not particularly preferred embodiments, the pharmaceutical compositions described in documents [1] and [2] can also comprise one or more polar excipients, such as ethanol, which are described as having the effect of solubilising the surfactant in the propellant and/or of inhibiting the deposition of particles of active ingredient on the surfaces of the canister.

In contrast, the pharmaceutical compositions described in document [3] do not comprise a surfactant for the reason that the surfactants would not be desirable and that it would be of interest to form a stable suspension without the use of a surfactant. Document [3] specifies that the use of the propellant gas R-152a allows to prepare pharmaceutical compositions that are free of surfactants and of polar excipients and that nevertheless have good pharmaceutical performance when they are delivered using a drug administration device such as a metered-dose inhaler (MDI).

However, the inventors observed that pharmaceutical compositions consisting only of salbutamol sulphate and of R-152a did not allow to obtain the expected aerosolization performance.

It is therefore on the basis of this observation and in the interest of constantly improving the aerosolization properties and, consequently, the therapeutic properties conferred by the pharmaceutical compositions intended for the treatment of respiratory disorders that the present invention is based.

### DISCLOSURE OF THE INVENTION

This goal as well as others are achieved, first of all, by a pharmaceutical composition of the aforementioned type, that is to say which comprises an active ingredient based on salbutamol and 1,1-difluoroethane as propellant gas.

According to the invention, the pharmaceutical composition consists of the following compounds:
(a) an active ingredient based on a pharmaceutically acceptable salbutamol salt,
(b) 1,1-difluoroethane (R-152a), and
(c) ethanol.

The inventors observed that, in an unexpected and surprising manner, a pharmaceutical composition that only comprises a salbutamol salt, R-152a and ethanol allows to achieve aerosolization performance much higher than that of a pharmaceutical composition only comprising a salbutamol salt and R-152a as described in document [3], this remarkable aerosolization performance furthermore being stable over time.

This result is even more unexpected since it goes against the teachings of documents [1] to [3] which recommend limiting, or even avoiding, implementing ethanol in the pharmaceutical compositions containing salbutamol sulphate and R-152a. These documents report in particular that ethanol can cause an unacceptable irritation of the mouth and of the throat, in particular in young patients, and/or cause a coarse spraying of the pharmaceutical compositions characterised by droplet sizes that are too large to allow to obtain acceptable penetration into the deep bronchioles of the lung.

In an advantageous variant of the pharmaceutical composition according to the invention, the active ingredient (a) is a salbutamol salt.

In a preferred variant of the invention, this active ingredient (a) is salbutamol sulphate.

The active ingredient (a) is advantageously in the form of particles, the size of which is adapted to a delivery by inhalation of the pharmaceutical composition in which it is contained. Conventionally, the median diameter of the particles of active ingredient (a), routinely noted as Dv₅₀, is less than or equal to 6 μm, which means that at least 50% by volume of the particles of active ingredient (a) have a diameter less than or equal to 6 μm.

This median diameter of the particles of active ingredient (a) is advantageously less than or equal to 5 μm, preferably between 0.5 μm and 5 μm and, more preferably, between 1 μm and 4 μm.

In a variant of the composition according to the invention, the mass proportion of active ingredient (a) is between 0.05% and 0.5% by mass relative to the total mass of the pharmaceutical composition. This mass proportion is advantageously between 0.1% and 0.4% by mass and, preferably, between 0.2% and 0.35% by mass relative to the total mass of the pharmaceutical composition.

In a variant of the composition according to the invention, the mass proportion of 1,1-difluoroethane (b) is between 89.5% and 99.9% by mass relative to the total mass of the pharmaceutical composition.

In another variant, the mass proportion of 1,1-difluoroethane (b) is between 94.5% and 99.9% by mass relative to the total mass of the pharmaceutical composition. This mass proportion is advantageously between 96.6% and 99.7% by mass and, preferably, between 97.65% and 99.3% by mass relative to the total mass of the pharmaceutical composition.

In a variant of the composition according to the invention, the mass proportion of ethanol (c) is between 0.05% and 10% by mass relative to the total mass of the pharmaceutical composition.

In another variant, the mass proportion of ethanol (c) is between 0.05% and 5% by mass relative to the total mass of the pharmaceutical composition.

The aerosolization performance of the pharmaceutical composition according to the invention can be reached with a relatively low mass proportion of ethanol, which does not present any danger for the health of the patient, even young.

The mass proportion of ethanol can advantageously be between 0.2% and 3% by mass and, preferably, between 0.5% and 2% by mass relative to the total mass of the pharmaceutical composition.

The present invention relates, secondly, to a pharmaceutical composition for use in the treatment of patients suffering or liable to suffer from respiratory disorders.

According to the invention, this pharmaceutical composition, which is used in the treatment of respiratory disorders, is as defined above, that is to say that it consists of the following compounds:
(a) an active ingredient based on a pharmaceutically acceptable salbutamol salt,
(b) 1,1-difluoroethane (R-152a), and
(c) ethanol.

The features described above in relation to the pharmaceutical composition and, in particular, the features relative to the active ingredient as well as to the mass proportions of the various compounds forming this pharmaceutical composition are of course applicable to the present use in the treatment of respiratory disorders.

Such respiratory disorders can be asthma or chronic obstructive pulmonary disease (COPD).

In the context of the present invention, the patients can be treated by the administration of a quantity effective from a therapeutic point of view of a pharmaceutical composition as defined above.

The present invention relates, thirdly, to a canister comprising a pharmaceutical composition as well as to a metered-dose inhaler comprising such a canister.

According to the invention, this pharmaceutical composition is as defined above, that is to say that it consists of the active ingredient (a), R-152a as propellant gas and ethanol, wherein the features relative to these compounds can be taken alone or in combination.

The present invention relates, fourthly, to the use of a pharmaceutical composition and/or of a canister as defined above in a metered-dose inhaler (MDI), such a device being conventionally used to deliver pharmaceutical compositions comprising an active ingredient based on salbutamol or on a pharmaceutically acceptable salt thereof.

Other features and advantages of the invention will be clearer upon reading the following additional description, which relates to examples of pharmaceutical compositions as well as to the evaluation of their in vitro aerosolization performance, two pharmaceutical compositions, noted as C4 and C4′, being according to the invention, the others being comparative pharmaceutical compositions according to the teachings of documents [1] and [3], noted as C1 to C3 and C3′.

### BRIEF DESCRIPTION OF THE FIGURES

FIG. 1 shows the graphs illustrating the deposited fraction of salbutamol particles (expressed in %) coming from doses or puffs of the pharmaceutical compositions C1 to C4 as measured at T0, as a function of the stages of the NGI pharmaceutical impactor.

FIGS. 2A, 2B and 2C show the graphs illustrating the deposited fraction of salbutamol particles (expressed in %) coming from doses or puffs of the pharmaceutical compositions C3′ and C4′ as respectively measured at T0, at T3M and at T6M, as a function of the stages of the NGI pharmaceutical impactor.

FIG. 3 reproduces the graphs of FIGS. 2A, 2B and 2C illustrating the deposited fraction of salbutamol particles (expressed in %) coming from the doses of the pharmaceutical compositions C4′ according to the invention as measured at T0, at T3M and at T6M, as a function of the stages of the NGI pharmaceutical impactor.

FIG. 4 reproduces the graphs of FIGS. 2A, 2B and 2C illustrating the deposited fraction of salbutamol particles (expressed in %) coming from the doses of the comparative pharmaceutical composition C3′ as measured at T0, at T3M and at T6M, as a function of the stages of the NGI pharmaceutical impactor.

### DETAILED DISCLOSURE OF SPECIFIC EMBODIMENTS

### Example 1

The aerosols that were subjected to the tests were manufactured with the same batches of compounds, of canisters made of aluminium and of metering valves.

Four pharmaceutical compositions C1 to C4 were prepared using the following quantities of salbutamol sulphate (median diameter of approximately 2 μm), of oleic acid and of ethanol mentioned in table 1 below.

TABLE 1

| | Salbutamol | Oleic acid | Ethanol |
|---|---|---|---|
| Composition | sulphate (mg) | (mg) | (mg) |
| C1 | 30.125 +/− 2.5% | 28.438 +/− 2.5% | 0 |
| C2 | 30.125 +/− 2.5% | 34.125 +/− 2.5% | 0 |
| C3 | 30.125 +/− 2.5% | 0 | 0 |
| C4 | 30.125 +/− 2.5% | 0 | 113.750 +/− 2.5% |

When applicable, oleic acid or ethanol was first introduced manually into four distinct series of canisters followed by salbutamol sulphate.

A metering valve was then crimped onto each of the canisters with a suitable equipment, then R-152a propellant gas was introduced using a suitable equipment via the metering valve to reach a total mass of pharmaceutical composition of 11375 mg.

The aerosols thus packaged were then stored in a so-called inverted position (valve downwards) during a quarantine period of at least one week.

At the end of this quarantine period, an aerodynamic particle size distribution (APSD) measurement test was carried out.

This test at T0, which consists of evaluating the aerodynamic size of the particles of active ingredient exiting the valve, was carried out via a multi-stage pharmaceutical impactor allowing an approximate modelling of the bronchial tree. In the present case, the pharmaceutical impactor used was the Next Generation Impactor (NGI) that corresponds to apparatus E of the European Pharmacopoeia.

More particularly, the tests were carried out at a flow rate of 30 L/min, by expulsing 5 doses of each of the compositions C1 to C4 into the NGI impactor.

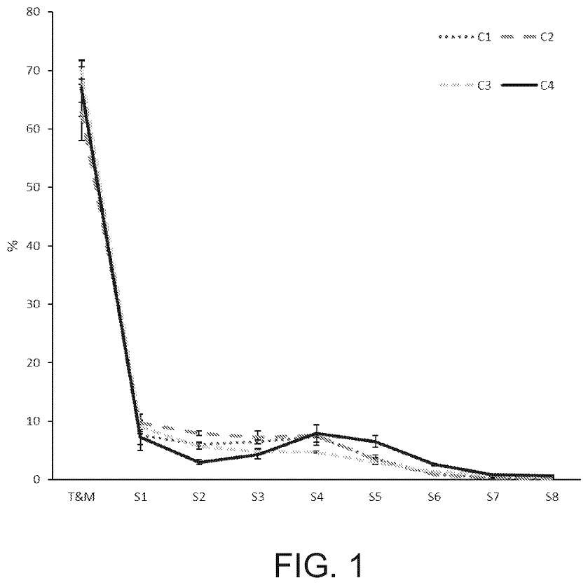

The graph of FIG. 1 shows the fraction of salbutamol deposited in the throat and in the mouth (noted as T&M) and on each of the stages of the impactor (noted as S1 to S8).

To ensure an optimal therapeutic efficacy, the fraction deposited on stages 3 to 6, noted as S3 to S6, and more particularly on S4 and S5, must be maximised.

FIG. 1 shows that the pharmaceutical composition C4 according to the invention, which comprises 1% by mass of ethanol, effectively allows to optimise this therapeutic efficacy since the fraction of salbutamol deposited on these stages S3 to S6, and in particular on the stages S4 and S5, is very clearly greater than the fractions of salbutamol deposited on these same stages using the comparative pharmaceutical compositions C1 to C3, this phenomenon being more marked using the comparative pharmaceutical composition C3. With regard to the data obtained with the comparative pharmaceutical compositions C1 and C2, it is observed that the implementation of the pharmaceutical composition C4 according to the invention allows a displacement of the fine particles from the stage S3 to the stage S4 and especially to the stage S5 which are smaller in size of fine particles.

This observation is even more surprising since:
on the one hand, the comparative pharmaceutical compositions C1 and C2 respectively comprise mass proportions of 0.25% by mass and 0.3% by mass that are within the preferred range of 0.2% by mass to 1.0% by mass of surfactant, in this case the oleic acid, taught by document [1], and
on the other hand, the comparative pharmaceutical composition C3 comprising neither surfactant nor ethanol is described by document [3] as having good pharmaceutical performance.

The therapeutic performance of the pharmaceutical composition according to the invention is, moreover, corroborated by the data of fraction of fine particles reported in table 2 below, a table in which the quantities of oleic acid or of ethanol present in the pharmaceutical compositions C1, C2 and C4 are reported in mass proportion (% by mass).

TABLE 2

| | Oleic acid | Ethanol | Fraction of fine |
|---|---|---|---|
| Composition | (% by mass) | (% by mass) | particles (in %) |
| C1 | 0.25 | 0 | 15 |
| C2 | 0.3 | 0 | 16 |
| C3 | 0 | 0 | 13 |
| C4 | 0 | 1.00 | 21 |

### Example 2

As in example 1, the metered-dose inhalers were prepared with the same batches of compounds, of canisters made of aluminium and of metering valves, according to an identical operating protocol.

In a first step, a metering valve was crimped onto each of the canisters with a suitable equipment.

In a second step, two distinct series of metered-dose inhalers were filled, using a pilot equipment and in two steps, by introduction via the metering valve:
of a concentrated suspension comprising 30.125 mg of salbutamol sulphate (median diameter of approximately 5 μm), a reduced quantity of propellant gas R-152a and, when applicable, the mass proportion of ethanol indicated in table 3 below, relative to the total mass of the pharmaceutical composition, then
of a sufficient quantity of R-152a propellant gas to reach a total mass of pharmaceutical composition of 9.57 g.

TABLE 3

| Composition | C3′ | C4′ |
|---|---|---|
| Ethanol (% by mass) | 0 | 1.00 |

Three series of aerodynamic particle size distribution (APSD) measurement tests were carried out at a flow rate of 30 L/min, by expulsing 5 doses of each of the pharmaceutical compositions C3′ and C4′ into the NGI impactor, as in example 1 above.

A first series of aerodynamic particle size distribution measurement tests was carried out on the pharmaceutical compositions C3′ and C4′ as obtained at T0, that is to say at the end of the quarantine period mentioned in example 1.

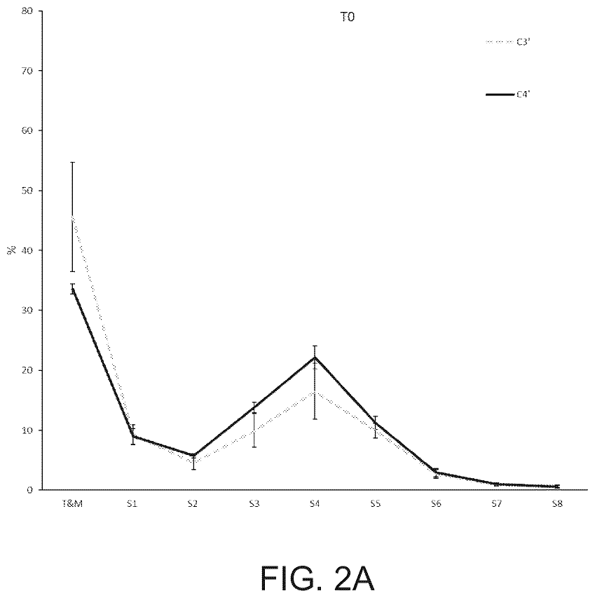

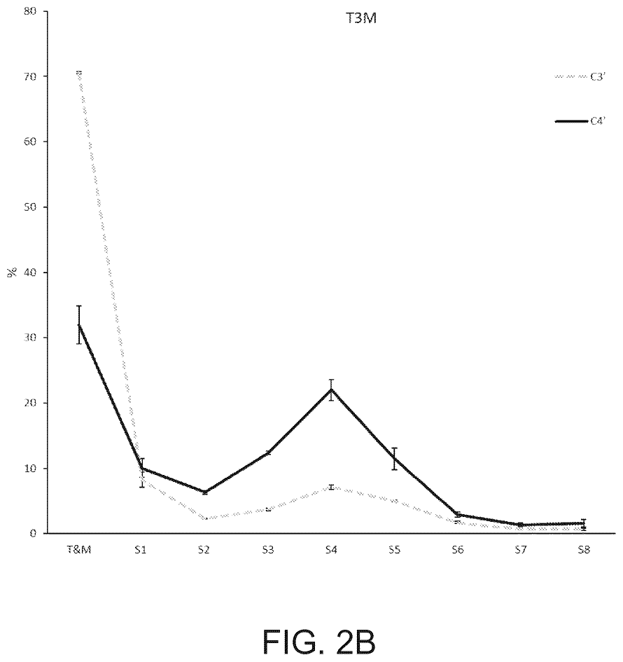

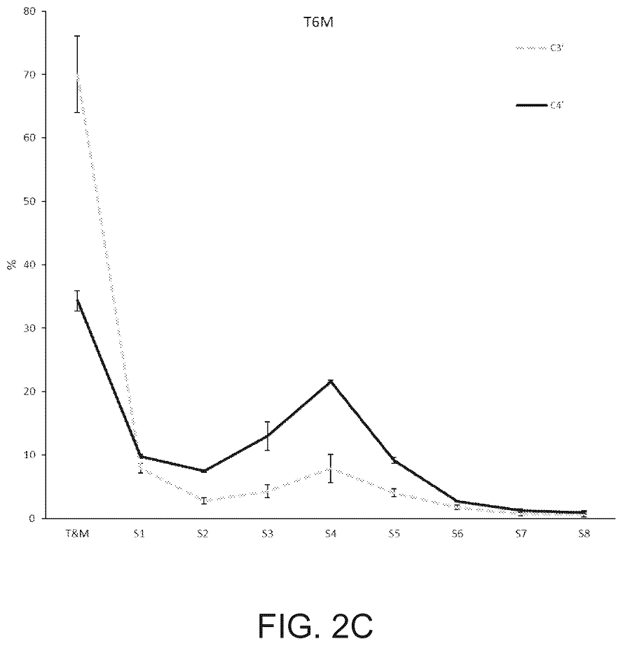

The results of this first series of tests at T0 are reported in FIG. 2A.

A second series of aerodynamic particle size distribution measurement tests was carried out on these same pharmaceutical compositions C3′ and C4′ as obtained at T3M, that is to say after a storage of the metered-dose inhalers comprising said compositions C3′ and C4′ for a period of three months starting from T0, these metered-dose inhalers being placed, during these three months, in an inverted position (valve downwards) in respective temperature and relative humidity conditions of 40° C. and 75% which are compliant with the directives of the International Council for Harmonisation for Pharmaceutical Quality (ICH Q1 Stability Guidelines).

The result of this second series of tests at T3M are reported in FIG. 2B.

A third series of aerodynamic particle size distribution measurement tests was carried out on these same pharmaceutical compositions C3′ and C4′ as obtained at T6M, that is to say after a storage of the metered-dose inhalers comprising said compositions C3′ and C4′ for a period of six months starting from T0, these metered-dose inhalers being placed, during these six months, in an inverted position and in the temperature and relative humidity conditions described in the previous paragraph.

The results of this third series of tests at T6M are reported in FIG. 2C.

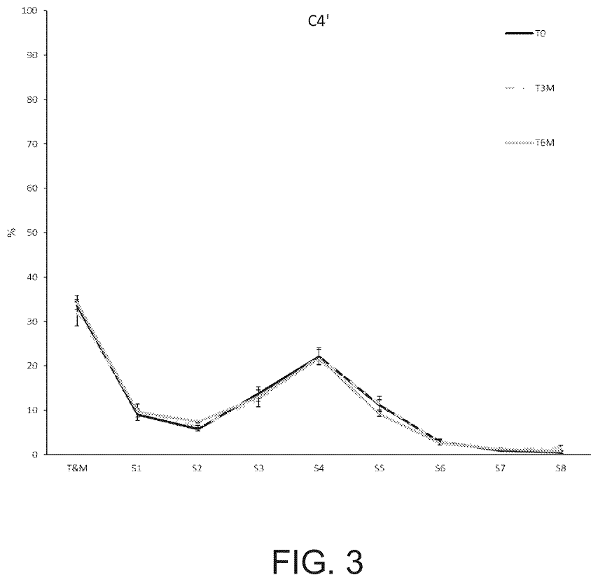

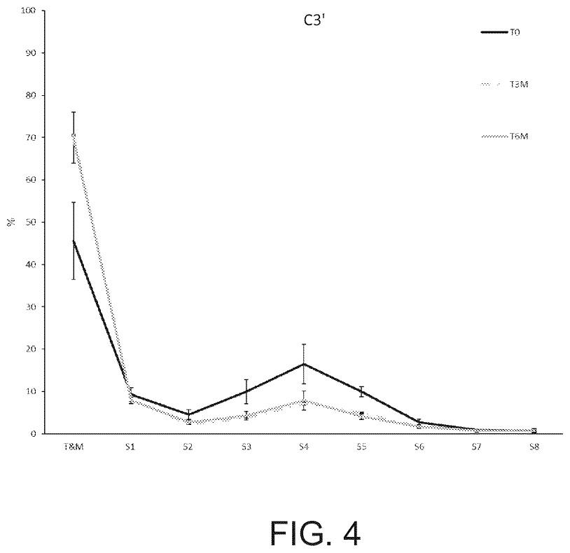

FIGS. 3 and 4 bring together the graphs of FIGS. 2A to 2C obtained with the pharmaceutical composition C4′ according to the invention (FIG. 3) and with the comparative pharmaceutical composition C3′ (FIG. 4). It is specified that this comparative pharmaceutical composition C3′ is consistent with the teaching of document [3].

The graphs of FIGS. 2A to 2C, 3 and 4 show the fractions of salbutamol deposited, on the one hand, at the throat and mouth (T&M) and, on the other hand, on each of the eight stages of the impactor (noted as S1 to S8).

To ensure an optimal therapeutic efficacy, the fractions deposited on the stages S3 to S6 must be maximised and the fraction deposited at T&M minimised.

FIGS. 2A, 2B and 2C show that the pharmaceutical composition C4′ according to the invention, which comprises 1% by mass of ethanol, effectively allows to optimise this therapeutic efficacy.

On the one hand, with reference to FIG. 2A, it is noted that the sum of the salbutamol fractions deposited at T0 on the stages S3 to S6 using the pharmaceutical composition C4′ is significantly greater than the sum of the salbutamol fractions deposited at T0 on these same stages S3 to S6 using the comparative pharmaceutical composition C31. This observation is even more marked if reference is made to the graphs of FIGS. 2B and 2C.

On the other hand, and still with reference to FIG. 2A, it is observed that the salbutamol fraction deposited at T0 using the pharmaceutical composition C4′ according to the invention at T&M is approximately 35% and thus much less than the salbutamol fraction deposited at T0 using the comparative pharmaceutical composition C3′, which is approximately 45%. If reference is made to FIGS. 2B and 2C, it is observed that this rate of 35% is preserved at T3M and T6M with the pharmaceutical composition C4′ according to the invention whereas it increases to reach values of approximately 70% with the comparative composition C3′.

FIG. 3 shows that the pharmaceutical composition C4′ according to the invention maintains this optimised therapeutic efficacy over time, even after six months of storage at 40° C. and 75% relative humidity. Indeed, the graphs of this FIG. 3 are practically superimposable, reflecting the fact that the sum of the salbutamol fractions deposited on the stages S3 to S6 as well as the salbutamol fraction deposited at T&M are similar, or even identical, at T0, T3M and T6M. In other words, the pharmaceutical composition C4′ according to the invention is characterised by an aerosolization performance stable over time.

On the contrary, if reference is made to FIG. 4, it is observed that the therapeutic efficacy is highly degraded for the comparative pharmaceutical composition C3′ at least already after three months of storage at 40° C. and 75% relative humidity (T3M) and, a fortiori, at T6M.

### BIBLIOGRAPHY

[1] WO 2013/054137 A1
[2] WO 2014/170689 A1
[3] WO 2013/054135 A1
[4] US 2007/041911 A1
[5] US 2021/244688 A1

What is claimed is:

1. Pharmaceutical composition consisting of the following compounds:
(a) salbutamol sulphate as active ingredient,
(b) 1,1-difluoroethane (R-152a), and
(c) ethanol.

2. Pharmaceutical composition according to claim 1, wherein the mass proportion of active ingredient (a) is between 0.05% and 0.5% by mass, advantageously between 0.1% and 0.4% by mass and, preferably, between 0.2% and 0.35% by mass relative to the total mass of the pharmaceutical composition.

3. (canceled)

4. Pharmaceutical composition according to claim 1, wherein the mass proportion of 1,1-difluoroethane (b) is between 89.5% and 99.9% by mass, in particular between 94.5% and 99.9% by mass, advantageously between 96.6% and 99.7% by mass and, preferably, between 97.65% and 99.3% by mass relative to the total mass of the pharmaceutical composition.

5. Pharmaceutical composition according to claim 1, wherein the mass proportion of ethanol (c) is between 0.05% and 10% by mass, in particular between 0.05% and 5% by mass, advantageously between 0.2% and 3% by mass and, preferably, between 0.5% and 2% by mass relative to the total mass of the pharmaceutical composition.

6. Pharmaceutical composition according to claim 1 for use in the treatment of respiratory disorders, such as asthma or chronic obstructive pulmonary disease (COPD).

7. Canister comprising a pharmaceutical composition according to claim 1.

8. Metered-dose inhaler (MDI) provided with a canister according to claim 7.

9. Use of the pharmaceutical composition according to claim 1 in a metered-dose inhaler (MDI).

10. Use of the canister according to claim 7 in a metered-dose inhaler (MDI).
